(19) United States
(12) Patent Application Publication
(10) Pub. No.: US 2021/0186803 A1
(43) Pub. Date: Jun. 24, 2021

(54) VIBRATORY INFRARED OLIGODYNAMIC APPARATUS AND MUSCULOSKELETAL PAIN TREATMENT METHOD USING SAME

(71) Applicant: ADM TRONICS UNLIMITED INC., Northvale, NJ (US)

(72) Inventor: Andre DiMino, Woodcliff Lakes, NJ (US)

(21) Appl. No.: 17/163,226

(22) Filed: Jan. 29, 2021

Related U.S. Application Data
(60) Provisional application No. 62/927,262, filed on Oct. 29, 2019.

(51) Int. Cl. A61H 23/02

(52) U.S. Cl. CPC A61H 23/0263; A61H 2201/10

(57) ABSTRACT

The present invention relates to an apparatus and a method for applying combined noninvasive modalities such as vibration modalities and phototherapeutic modalities, either singularly or in combination, through an oligodynamic surface to modify the physiological response of a person to treat pain and discomfort from musculoskeletal disorders, and conditions such as wound healing, muscle atrophy and other medical conditions and diseases.

### CROSS REFERENCE TO RELATED APPLICATIONS

This patent application claims the benefit of U.S. Patent Provisional Application No. 62/927,262, filed on Jan. 29, 2020, the entire disclosure of which is incorporated herein by reference.

### FIELD OF THE INVENTION

The present invention relates to a vibratory infrared oligodynamic apparatus; and, more particularly, to an apparatus and a therapeutically beneficial method for using a vibratory infrared oligodynamic apparatus for non-invasive treatment of musculoskeletal pain and discomfort, soft tissue diseases, injuries, and poor blood circulation conditions in anatomical areas of humans and animals.

### DESCRIPTION OF RELATED ARTS

Vibration therapy has been in wide use for the treatment of painful and discomfort of joints such as chronic and acute musculoskeletal disorders which include, but are not limited to, osteoarthritis, tendinitis, bursitis, epicondylitis, temporomandibular joint dysfunction, and sports injuries. Vibration therapy is administered with a wide array of apparatus formats. Vibration serves to distract pain signals, limiting their impact. Compressing the vibration at the treatment target area enhances blood circulation and simultaneously limits discomfort. The sensation of vibration soothes pain receptors and increases warmth at the treatment site. This benefits muscles, tendons and ligaments that can strengthen joints and helps recovery from injury, chronic, pain and discomfort of musculoskeletal disorders. Slight but continuous sensation may also mask pain by blocking or tricking signals being sent to the brain.

Additionally, phototherapy, for example, utilizing light emitting diodes of certain frequencies, commonly referred to as LEDs, cause bioreactions by cellular photoactivation without heat or damage. Although visible and near-infrared light energy induce the same tri-stage process in target cells, namely photon absorption, intracellular signal transduction and final cellular photoresponse, it should be noted that both wavebands have different primary targets and photoreactions in target ceils. Visible tight is principally a photochemical reaction, acting directly and mostly on cytochrome-c oxidase, which is the end terminal enzyme in the cellular mitochondrial respiratory chain, and mainly responsible for inducing adenosine triphosphate (ATP) synthesis, which is the fuel of the cell and the entire metabolism.

Infrared light induces a primary photophysical reaction in the cell membrane thereby kick-starting the cellular membrane transport mechanisms such as the Na K pump, which in turn induces as a secondary reaction, the same photochemical cascade as seen with visible light, so the end result is the same even though the target is different.

The field in which infrared therapy has been most, used for involves pain control, with pain of almost all etiologies responding well. For example, phototherapy significantly reduced both acute and chronic pain in professional athletes. Researchers have been using LED in the control of herpes zoster pain for some time, as well as for intractable postherpetic neuralgia. This and other chronic pain entitles have been historically very hard to control, but the efficacy of LED phototherapy has been well recognized. LED phototherapy can help nerve regeneration, so it has been used for spinal cord injuries, and many different types of neurogenic abnormality. There is strong evidence for its use in guided bone regeneration and treatment of wounds by inducing faster and better-organized healing than in the control of unirradiated wounds

In addition, the oligodynamic effect of metals relates to the toxic effect of metal ions on living cells, algae, molds, spores, fungi, viruses, prokaryotic and eukaryotic microorganisms. This has been confirmed to occur even in relatively low concentrations of the presence of the metals.

There have been numerous studies of the effect of metals, especially certain heavy metals such as silver and copper, as antibacterial and germicidal surfaces. In oligodynamic action, metal ions concentrate on the surface of a living object (bacteria, algae, and so on), which causes blocking of the free carbonyl and sulfhydryl groups of the surface structures. Oligodynamic action extends to enzyme systems and even to the activity of inorganic catalysts. The toxic effect of negative ions, which are capable of inactivating enzyme systems and whose activity depends on the presence of metal atoms, is sometimes also included among the oligodynamic actions. An indispensable condition for the oligodynamic action of metals is their ability to pass into the ionized state.

### SUMMARY OF THE INVENTION

Various embodiments according to the present disclosure pertain to an apparatus which combines a vibratory and phototherapy modality with the oligodynamic properties of a metal treatment surface that is specifically designed to allow for easier treatment of anatomical areas, and a method for using the apparatus to treat musculoskeletal disorders and discomfort with a multifaceted therapeutic approach are provided.

Embodiments of the present disclosure relate to the treatment of musculoskeletal disorders using a combination of noninvasive treatment modalities implemented with a treatment surface providing the beneficial effect from the oligodynamic properties of metals. The present invention also relates to a method for applying combined noninvasive modalities through an oligodynamic surface to modify the physiological response of a person to treat pain and inflammation from musculoskeletal disorders, and for the treatment of soft tissue conditions such as wound healing, muscle atrophy and other musculoskeletal conditions and diseases. The present invention further involves various combinations of vibratory and phototherapy modalities with an oligodynamic treatment surface in a small portable apparatus for ease of application in everyday life. The present invention also relates to a combination of phototherapy modalities with an oligodynamic treatment surface allowing for the cellular benefits of the phototherapy with the biocidal and antibacterial properties of an oligodynamic metal surface. The present invention can also use the vibratory and phototherapy modalities individually with an oligodynamic treatment surface. Furthermore, the present invention may also include one or more of the vibratory and phototherapy modalities with an oligodynamic treatment surface and does not need to include all of the modalities.

According to an embodiment of the present disclosure the intensity of the vibration can be varied. In addition, a user can selectively turn on and off the vibratory and LED functions in the apparatus. In the case of the LEDs, a user can selectively choose different frequencies of the LEDs for example, 940 nm; 880 nm; 830 nm; 660 nm; and 630 nm, or a combination of the frequencies, however, other frequencies and combinations can be used.

In some anatomical areas it is difficult to administer or apply a therapeutic, non-invasive modality to treat a painful condition. Accordingly, various embodiments of the present disclosure provide an apparatus which is lightweight, portable, and easy to use.

According to an embodiment of the present disclosure, a method is disclosed which operates to change the physiological parameters underlying a disease, injury or condition by employing the beneficial effects of a combination of nan-invasive therapies, such as, vibration, phototherapy, and a treatment surface with the oligodynamic properties of metal. The method more particularly includes various combinations of vibratory and phototherapy modalities with and through a treatment surface comprised of an oligodynamic metal.

An apparatus and method according to the present invention relate to a therapeutically beneficial method for non-invasive treatment of musculoskeletal pain and discomfort, soft tissue diseases, injuries and other painful conditions. This inventive method operates to change the physiological parameters underlying a disease, injury or condition by employing the beneficial effects of a combination of non-invasive therapies with the oligodynamic properties of metal. An embodiment of the apparatus according to the present disclosure can include the combined treatment modalities of vibratory, phototherapy, and oligodynamic modalities in a portable device, and can be applied for treatment by a user when and where it is needed. Furthermore, in another embodiment of the present disclosure, a user may choose either the vibratory treatment modality, the phototherapy treatment modality, or both modalities, to be used in combination with the treatment benefits of the oligodynamic surface of the apparatus.

It is another object of the present invention to provide an improved method for increasing blood flow to affected tissues by the applying the effects of vibration and phototherapy through an oligodynamic surface.

It is yet another object of the invention to provide an improved method for treatment of musculoskeletal pain and discomfort related to conditions such as osteoarthritis, tendinitis, bursitis, epicondylitis, temporomandibular joint dysfunction, and sports injuries by employing the beneficial effects of a combination of non-invasive therapies with the oligodynamic properties of metal.

It is a still further object of the invention to provide an improved means for the treatment of soft tissue disease, injury or conditions such as wounds, muscle atrophy, nerve regeneration and other internal soft tissue disease, injury or conditions by employing the beneficial effects of a combination of non-invasive therapies with the oligodynamic properties of metal.

It is a further object of this invention to provide an apparatus that allows for the interchangeability of the phototherapy component to provide a means to alter the frequency of the output for differing therapeutic effects by removing one set of phototherapy emitters and replacing with another set of different wavelength.

It is also an object of the invention to provide a means of targeting the phototherapy portion of the therapeutic output by providing a variable selection of portions of the LED array that are configured to reach various anatomical areas.

It is a further object of the invention to provide a means for selecting all or various portions of LED output for different conditions of anatomical areas by use of controls on the apparatus, or by wireless connection of the therapeutic apparatus with a smartphone, tablet or other communications device thereby allowing for ease of operation without the necessity of reaching for the apparatus before, during or after use.

It is another object of this invention to provide for the recording of therapeutic sessions and communication of the use of the apparatus to physicians, caregivers or for the users own records through the internet, cloud, or any other type of communication network.

In addition, the oligodynamic effect of metals, especially heavy metals, relates to the biocidal and antibacterial properties of such metals when in contact with surfaces which may carry various microorganisms that, cause disease.

It is a further object of this invention to provide an apparatus that allows for the interchangeability of the phototherapy component to provide a means to alter the frequency of the output for differing therapeutic effects by removing one set of phototherapy emitters and replacing with another set of different wavelength.

It is a further object of this invention to provide an apparatus which can be used to treat animals as well as humans.

The above and other objects and advantages of the present invention will become apparent from the hereinafter set forth Brief Description of the Drawings and Detailed Description of the Invention.

It is intended that any other advantages and objects of the present invention that become apparent from the detailed description, drawings or illustrations contained herein are within the scope of the present invention.

### BRIEF DESCRIPTION OF THE DRAWINGS

The above and other objects and features of the present invention will become better understood with regard to the following description of embodiments of the present disclosure provided in conjunction with the accompanying drawings, in which:

FIGS. 1A end 1B respectively illustrate a side view and rear view of an apparatus according to an embodiment of the present disclosure;

FIGS. 2A and 2B respectively illustrate an angled upper perspective view and an angled lower perspective view of an apparatus according to an embodiment of the present disclosure;

FIGS. 3A and 38 respectively illustrate a rear view and a side view of internal components of an apparatus according to an embodiment of the present disclosure;

FIGS. 4A, 48, and 4C respectively illustrate a side view, a rear view and a bottom view of an oligodynamic base according to an embodiment of the present disclosure;

FIG. 5 illustrates additional internal components for the apparatus which allow communication with a wireless device according to an embodiment of the present disclosure;

FIG. 6A illustrates a front view of a mounting strap for the apparatus according to an embodiment of the present disclosure;

FIG. 6B illustrates a side view of a mounting strap for the apparatus according to an embodiment of the present disclosure; and

FIG. 7 illustrates a method for treatment of musculoskeletal pain and discomfort according to an embodiment of the present disclosure.

It Is intended that any feature of any embodiment of the present invention can be included as a feature in another embodiment of the present invention unless stated otherwise.

It is further intended that any other embodiments of the present invention that result from any changes in application or method of use or operation, method of manufacture, shape, size or material which are not specified within the detailed written description or illustrations and drawings contained herein, but are considered apparent to one skilled in the art, are within the scope of the present invention.

### DETAILED DESCRIPTION OF THE INVENTION

Hereinafter, various embodiments of the invention wilt be described in detail along with the accompanying drawings.

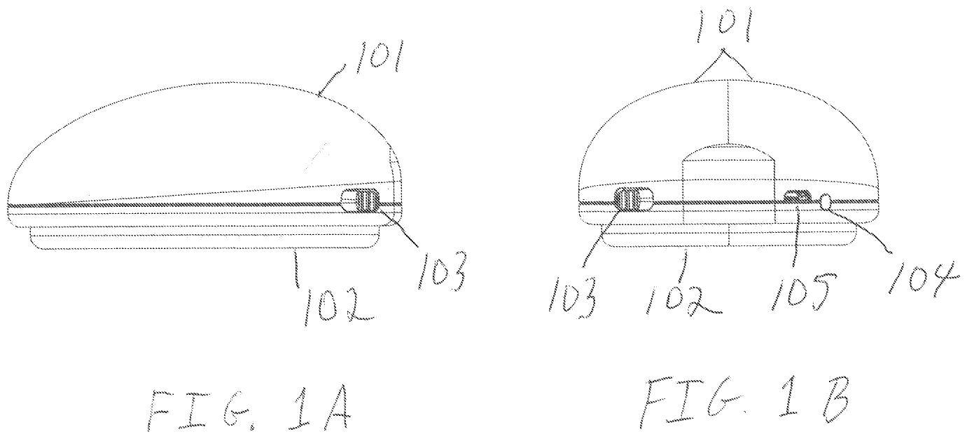

FIGS. 1A and 1B respectively illustrate a side view and rear view of an apparatus according to an embodiment of the present disclosure.

As illustrated in FIGS. 1A and 1B, an embodiment of the apparatus includes a housing 101 and an oligodynamic base 102. The housing further includes a multi-function switch 103 on a side of the housing as shown in FIG. 1A or on the rear of the housing as shown in FIG. 18, however, this is only for illustration purposes and the multi-function switch 103 can be positioned anywhere on the housing. Further illustrated in FIG. 1B is an on/off indicator 104 positioned on the rear of the housing and indicates whether or not the unit is powered on, however, this is only for illustration purposes and the on/off indicator 104 can be positioned anywhere on the housing. Additionally, as shown in FIG. 1B, a power port 105 is positioned on the rear of the housing, however, this is only for illustration purposes and the power port 105 can be positioned anywhere on the housing. The power port 105 can either be connected directly to a power source (not shown) for a power supply, such as an ac or dc power outlet, or can be used to charge an internal battery (not shown) which supplies power and is located under the housing 101.

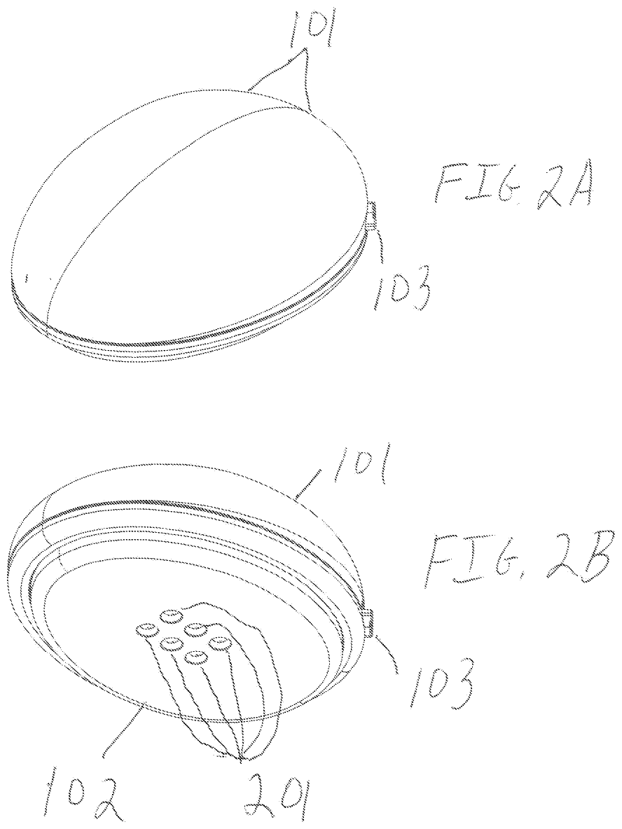

FIGS. 2A and 28 respectively illustrate an angled perspective upper view and an angled perspective lower view of an apparatus according to an embodiment of the present disclosure.

As illustrated in FIGS. 2A and 2B, an embodiment of the apparatus has a multi-function switch 103 positioned on a side of the housing 101. The multi-function switch 103 is configured to perform at least one of the following functions, turns a power supply to the apparatus on or off, controls the vibration speed of the apparatus, and allows a user to select at least one of a plurality of LEDs (not shown) having the same or various frequencies to be activated or deactivated. Furthermore, as shown in FIG. 28, the oligodynamic base 102 has a plurality of light exit outlets 201 through which light is emitted from the plurality of LEDs (not shown). Each LED corresponds to each light exit outlet in the plurality of light exit outlets 201. Each of the plurality of LEDs can either be positioned inside a corresponding light exit outlet or positioned in the housing 101 directly above a corresponding light exit outlet.

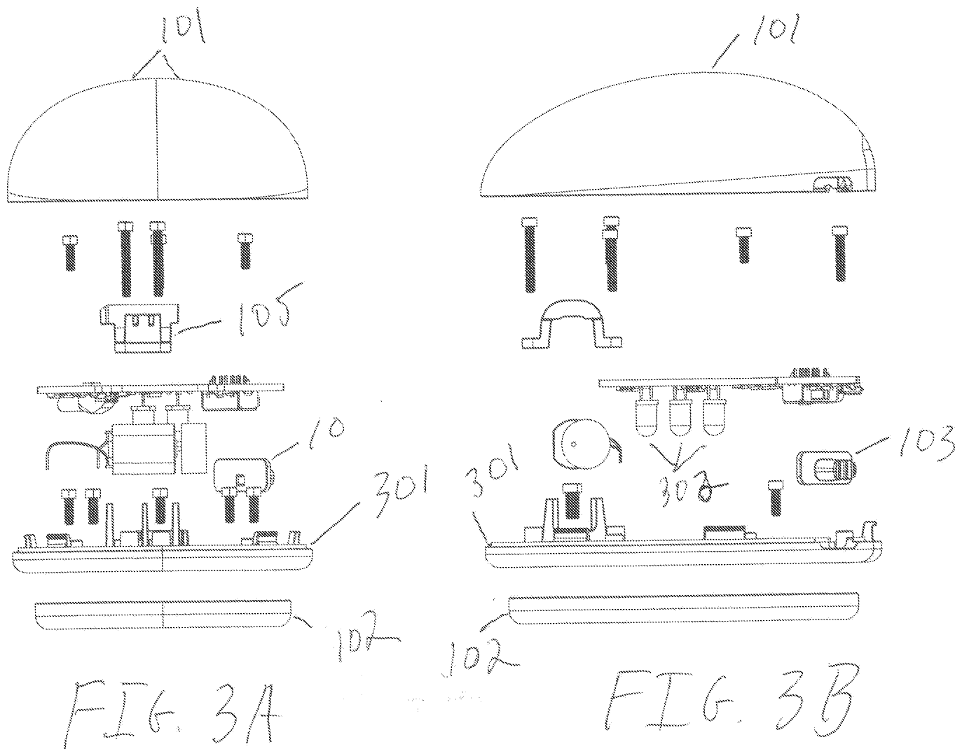

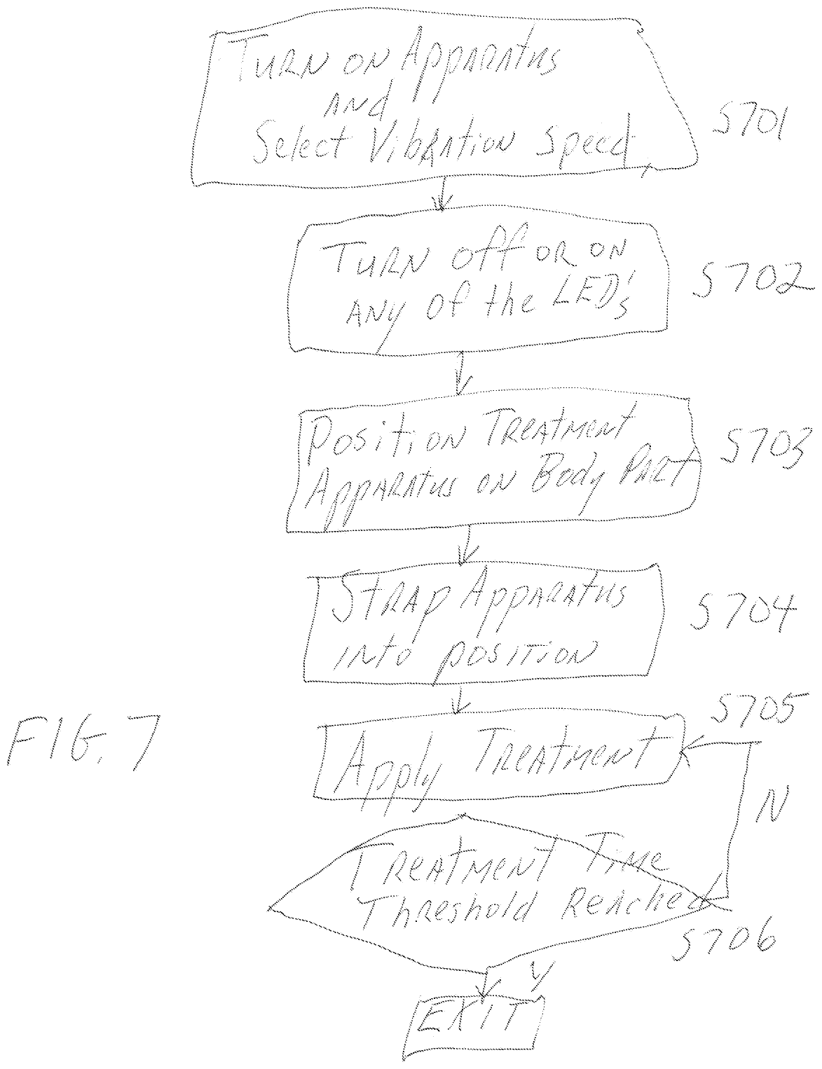

FIGS. 3A and 38 respectively illustrate a rear view and a side view of internal components of an apparatus according to an embodiment of the present disclosure.

As illustrated in FIGS. 3A and 3B, a housing 101 covers and protects internal components of the apparatus. The housing is connected to a circuit board 301 on which the internal components are affixed. The housing 101 and the circuit board 301 are further connected to an oligodynamic base 102 having a plurality of light exit cutlets (not shown).

The Internal components include but are not limited to a LED array board which includes a plurality of LEDs 302 and is interchangeable with other LED array boards which have the same frequencies, different frequencies, or a combination of the same frequencies and different frequencies. The plurality of LEDs 302 can output the same or various infrared (IR) LED frequencies for therapeutic use, which include 940 nm; 880 nm; 830 nm; 660 nm; and 63 nm, however other IR LED frequencies can be used. The plurality of LEDs 302 can either be ail of the same IR frequency or any combination of IR frequencies depending on a need of a user.

The plurality of LEDs 302 can be in the form of a LED array module (not shown) which can be detached and inserted into a LED slot (not shown) in the apparatus by a user. This advantageously allows a user to select a particular frequency setting of light waves which may be more effective in treating a particular type of disorder. In an embodiment, the LED array module can be removed from and inserted into a LED slot (not shown) on a side of the housing 101. In another embodiment, the LED array module can be fixed onto the oligodynamic base 102 such that the entire oligodynamic base can be exchanged with another oligodynamic base having a different LED array module with either the same oligodynamic base metal or a different oligodynamic base metal.

The internal components also include the multi-function switch 103, the on/off indicator 104, the power port 105, and preferably, an electric motor 303 configured with an offset counter weight, to generate and direct vibrations to the oligodynamic base 102. Tire speed of the vibrations at which the vibrations are generated are in the range of 30 to 465 vibrations per second however other vibration speeds can be used. Furthermore, the embodiments of the present disclosure are not limited to a motor with an offset counter weight and other motor configurations which generate equivalent vibration speeds can be used.

Using a weighted vibrating oligodynamic metal significantly enhances the pain-relieving action in treatment, of musculoskeletal conditions. The choice of an oligodynamic metal serves the purpose of providing the weight needed and, since the apparatus will be used on multiple painful target areas, advantageously avoids the transference of microorganisms from one area to another due to the germicidal, antimicrobial affects of the oligodynamic metal.

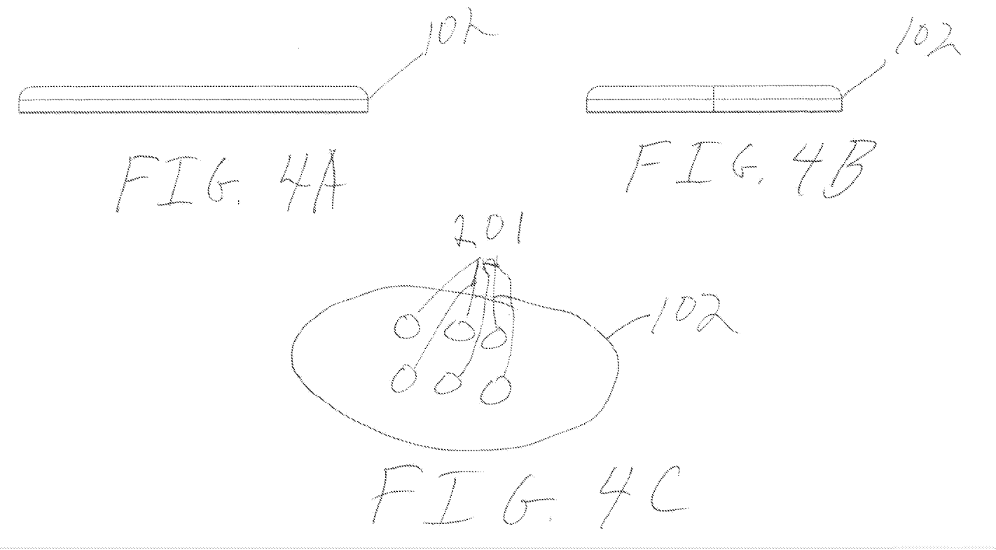

FIGS. 4A, 48, and 4C respectively illustrate a side view, a rear view and a bottom view of an oligodynamic base according to an embodiment of the present disclosure.

As illustrated in FIGS. 4A, 48, and 4C, the oligodynamic base 102 includes a plurality of light exit outlets 201. The plurality of LEDs 302 may be positioned either directly inside the plurality of light exit outlets 201 or inside the housing 101 and above the plurality of light exit outlets 201 so that IR light is emitted from a bottom of the apparatus opposite the housing 101.

The oligodynamic base 102 may be contoured so that it fits comfortably against a user's body or body part. The oligodynamic, base 102 may be constructed from oligodynamic materials, preferably at least one of Aluminum, Antimony, Cobalt, Copper, Gold, Lead, Molybdenum, Nickel, Silver, Zinc, and Zirconium. However, other materials which have oligodynamic properties may be used. Furthermore, the apparatus may be designed such that properties of the materials used for the oligodynamic base 102 are selected according to their efficacy in treating certain types of ailments.

Any oligodynamic metal may be used, for the best anti-microbial effect a metal with the highest antimicrobial effect would be most desirable. Silver is the highest, but due to the expense the next highest effectiveness is copper. Accordingly, a solid bar of copper 110 alloy may be employed. Copper 110 Alloy is the purest grade of oxygen-free copper containing 99.99% copper with a density of at least 1.5 g/cc preferably 8.93 g/cc and the lowest level of potential impurities may foe used.

In an embodiment of the present disclosure, a solid copper 110 alloy bar was machined to a desired shape and size for an intended hand-held treatment application for the apparatus. A contact surface area of approximately 6.54 square centimeters in an oblong oval shape allows for ergonomic ease of application to various musculoskeletal target areas on the body. It is also possible to use a copper 110 alloy surface covering another metal or non-metal material such as ceramic, so that the combination will yield the same weight but maintain the oligodynamic contact surface.

According to an embodiment of the present invention a solid copper 110 ally bar of 0.9525 cm thickness and 15.24 cm square area was machined to the shape and with the features for use on the apparatus to arrive at a finished weight of 275 grams of copper with an overall 0.635 cm thickness. This amount was determined to be the best efficacy in treating musculoskeletal pain due to its translation of the vibratory motor providing an effective deep treatment. Other thicknesses and weight of copper could be used but affects the specifications of the vibratory motor and the resulting desired efficacious massaging affect, as described below.

The apparatus utilizes a large mass copper base to maximize the kinetic energy of a vibrating massager with displacements that are in sub millimeter range and a frequency range that is low enough to be felt as a massaging motion.

Vibrations are created using an electric motor 303 spinning an unbalanced mass that is rigidly attached to the copper base.

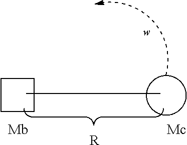

The diagram above shows a simplified representation where a counter mass “Mc” is spun around the copper base “Mb” with a radius “R” at an angular velocity “ω”. For the motor employed, according to an embodiment of the present invention, the radius of the weight is 8 mm.

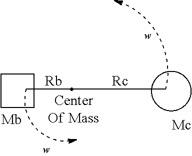

Since the copper base mass “Mb” is not fixed it will oscillate around a common center of mass. In the diagram above “Rb” is the distance between the rotational center of mass and the copper base mass “Mb” and “Rc” is the distance from the center of mass to the counter mass “Mc”.

The Reduced Hass Equation of two rigidly attached point bodies is used to determine “Rb” and “Rc”,

$$R = Rb + Rc$$

$$μ = \frac{Mb Mc}{Mb + Mc}$$

$$Rb = \frac{Mc R}{Mb + Mc} Rc = \frac{Mb R}{Mb + Mc}$$

Using the values for “Rb” and “Rc” the rotational moment of inertia “I” can then be calculated.

$$I = Mb {Rb}^{2} + Mc {Rc}^{2}$$

$$I = \frac{Mb {Mc}^{2} {R}^{2}}{{( Mb + Mc )}^{2}} + \frac{Mc {Mb}^{2} {R}^{2}}{{( Mb + Mc )}^{2}}$$

$$I = μ {R}^{2}$$

Deriving the tangential velocity “V” of the angular velocity “ω” using the equation

ω==V/R

The kinetic energy “K” of the system is.

K=½Iω²=½μV²

It is clear that a large copper base mass “Mb” maximizes the kinetic energy “K”, while reducing the amplitude “Rb” of vibrations on the base. This provides the effective vibratory massaging effect for soothing the musculoskeletal pain and enhancing blood flow to the area for the treatment.

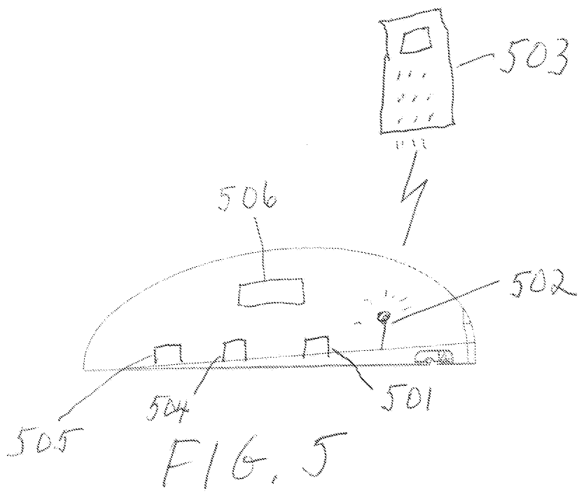

FIG. 5 illustrates additional internal components for the apparatus which allow communication with a wireless device according to an embodiment of the present disclosure.

Referring to FIG. 5, the internal components may further include one or more of a wireless transceiver 501 and antenna 502 for communicating various control settings and data regarding treatment times, vibration settings and LED selection settings with a wireless device 503 for example, a cell phone, tablet, smart phone, personal computer, and the like. The transceiver 501 and the antenna 502 may also be a single unit. The internal components may further include an one or more of a non-volatile memory 504 for retaining the various control settings and data within the apparatus and a volatile memory 505 for retaining temporary user data for example, a user's override of a stored control setting.

In an embodiment according to the present disclosure, the housing 101 can be configured with an external slot 506 which allows access to a LED array board so that, the LED array board can be easily removed and replaced with a different LED hoard having the same LED frequencies, different LED frequencies, or a combination of the same LED frequencies and different LED frequencies.

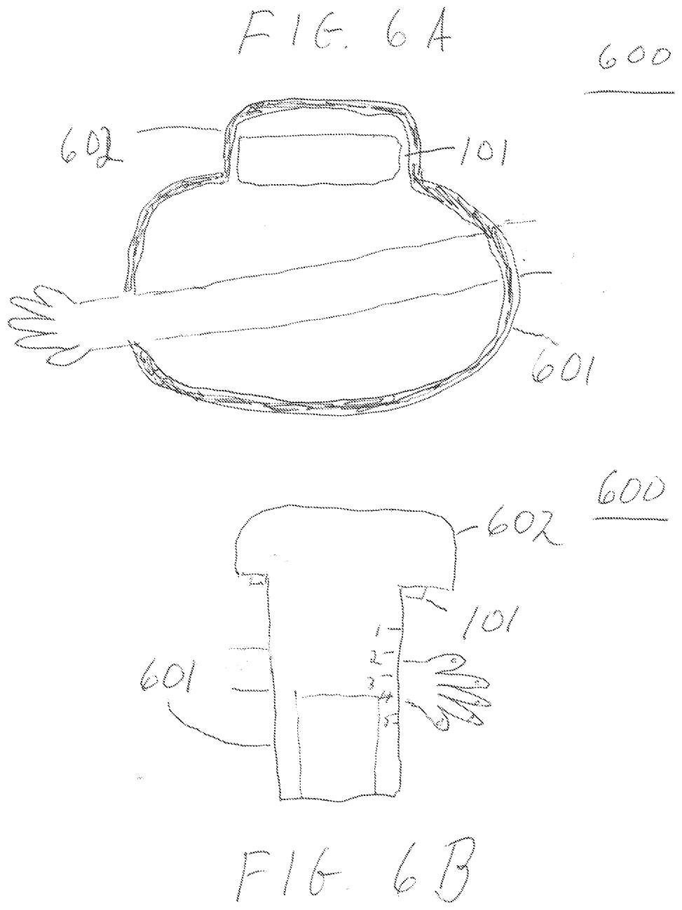

FIGS. 6A and 6B respectively illustrate a front view and a side view of a mounting strap for the apparatus according to an embodiment, of the present disclosure.

Referring to FIGS. 6A and 6B, in an embodiment, once an optimal treatment position is found by the user, a mounting strap 600 can be used to secure the apparatus to the user's body. The mounting strap 600 includes a pouch portion 602 in which the apparatus housing 101 can be inserted, anti an adjustable strap portion 603. The pouch portion 602 secures the apparatus to the mounting strap 600 to hold the apparatus in position against a user's body part, once the user determines the optimal position for relief of their pain. The adjustable strap portion 601 is calibrated with any of numerals, strap holes, symbols, or letters so that the user can control the amount of pressure applied against the user's body part to further enhance the effectiveness of the apparatus and enhance pain relief. The adjustable strap portion 601 can be adjusted to fit a user's body part by using any common adjustable fastener such as, Velcro®, a belt buckle, snaps, buttons, slip loop, and the like. Once an optimal pressure is determined by the user, the user can note the calibration so that the user can use the same calibration for subsequent pain relief treatments,

FIG. 7 illustrates a method for treatment of musculoskeletal pain and discomfort according to an embodiment of the present disclosure.

Referring to FIG. 7, in operation, a user turns on the apparatus and selects a vibration speed S701 which causes the oligodynamic base to vibrate and light from the LEDs to be emitted through the LED light exit outlets 201. In an embodiment, the user can optionally turn on or off at least one of the LEDs in the plurality of LEDs S702 depending on a user's needs. The user would position the apparatus on the treatment area sc that the oligodynamic base contacts the treatment area S703. The user would strap the apparatus in place. S704. The apparatus would be held in place and apply treatment to the treatment site. S705 When a treatment threshold time is reached S706, preferably for 3 to 8 minutes depending on the severity of the user pain, the treatment would end. While the present invention has been described with respect to various embodiments, it will be apparent to those skilied in the art that various changes and modifications may be made without departing from the scope and spirit of the disclosure as defined in the following claims.

What is claimed is:

1) An apparatus for treating musculoskeletal pain comprising:
a housing connected to a circuit board and an oligodynamic base, the oligodynamic base including a plurality of LED exit outlets;
an array of a plurality of LEDs affixed to the circuit board, each LED of the plurality of LEDs respectively corresponding to each of the plurality of LED outlets;
an electric motor with an offset counter weight, the electric motor being affixed to the circuit board, and configured to generate and direct vibrations to the oligodynamic base; and
a power supply configured to generate power for the apparatus,
wherein a light emitted from each of the plurality of LEDs passes through each of the plurality of LED exit outlets while the oligodynamic base is vibrating.

2) The apparatus of claim 1, further comprising a multi-function switch configured to perform at least one of powering the apparatus on or off, increasing or decreasing the vibration speed, activating or deactivating individual LEDs of the plurality of LEDS.

3) A method for treating musculoskeletal pain comprising:
powering on an apparatus configured to generate vibrations anti emit infrared LED light, and selecting a vibration speed;
activating or deactivating individual LEDs in a plurality of LEDS;
positioning the apparatus on a body part to be treated;
affixing the apparatus onto the body part by using an adjustable strap; and
treating the body part with the vibrations and the emitted infrared LED light until a treatment time threshold is reached.
